(19) United States
(12) Patent Application Publication
(10) Pub. No.: US 2026/0363905 A1
(43) Pub. Date: Nov. 19, 2026

(54) Anchored lacrimal caruncle implant for drug delivery and method

(71) Applicant: Bruce B. Becker, Malibu, CA (US)

(72) Inventor: Bruce B. Becker, Malibu, CA (US)

(21) Appl. No.: 19/126,607

(22) PCT Filed: Nov. 6, 2023
(86) PCT No.: PCT/US2023/036892

§ 371 Date: May 1, 2025

Related U.S. Application Data
(60) Provisional application No. 63/422,119, filed on Nov. 3, 2022.

(51) Int. Cl. A61F 9/00; A61K 9/00; A61K 45/06

(52) U.S. Cl. CPC A61F 9/0017; A61K 9/0051; A61K 45/06; A61F 2210/0004

(57) ABSTRACT

An implant (11) placed through the lacrimal caruncle (10) of a patient for prolonged delivery of a drug to the eye (1) for treating conditions such as glaucoma. The implant can include a drug-carrying depot which elutes the drug at a substantially predictable rate. Once depleted the depot can be recharged in vivo with an additional amount if the same or different drug. Various structural variations can adjust the implant's level of comfort, resistance to extrusion, bioabsorbability, flow rate of drug being delivered, and the delivery of the drug to specific locations on or in the eye.

### PRIOR APPLICATION

This application claims the benefit of U.S. Provisional Patent Application Ser. No. 63/422,119, filed 2022 Nov. 3, incorporated herein by reference.

### FIELD OF THE INVENTION

This invention relates to drug therapy and more particularly to the placement of a drug releasing implant through the lacrimal caruncle and surrounding tissue.

### BACKGROUND

Traditional ocular drug delivery has many short comings. Topical medications are used to treat many eye diseases, such as glaucoma, infection, and inflammation. However, non-compliance with the application of topical medications is a significant problem. Eyedrops must be instilled daily, often many times a day. Patient compliance is often inadequate leading to subtherapeutic dosing. This can lead to loss of vision and in many cases blindness.

This is especially an increasing problem in treating glaucoma. It is believed that glaucoma affects 2.2 million people in the United States and 67 million people worldwide. It is estimated that the number of glaucoma patients will increase by 50% in the next 15 years. Patients may fail or are unable to instill drops daily or to instill all of their daily drops. Many patients are unable to properly instill drops. Ineffective technique may lead to inadequate dosing or excessive use of drops resulting in side effects or costly waste of drops. In addition the peak effect of many drops is within two hours of being instilled. This leaves an inadequate effect for much of the time requiring frequent application of drops, further reducing compliance. Less than 1 to 5% of topically administered drops reach the aqueous humor. This results in a minimal effect since drops are only administered 1 to 4 times daily, rather than continuously.

Injections of medications are sometimes performed. However, this is painful, requires a doctor visit and has a short-lasting effect.

As a result, drug releasing implants have been proposed for drug therapy as disclosed in Tu, et al., U.S. patent Ser. No. 10/406,029. Such implants are intended to release medication that would flow over the eye, and would release medication over a significant period of time. Some types of implants can be removed when the drug is depleted and a new implant placed. Such implants typically consist of a retention structure and a drug core. Other types of implants are biodegradable and do not need to be removed when the drug has been entirely released. Unfortunately many implants may produce a burst release whereby much of the drug is released early and less is available for later release. Biodegradable implants may have a greater burst release than non-biodegradable implants.

Drug releasing implants may be placed on the surface of the eye or the surface of adjacent structures. Some implants are placed completely inside the eye. Such implants can release drug onto the surface of the eye or beneath the surface directly into the eye. The amount released and the region of drug release can make the coverage of the eye and penetration into the eye variable.

An implant placed in the superior conjunctival cul-de-sac is one such drug releasing implant. This implant has a high extrusion rate even with the use of adhesives. For example the Ocufit brand implant, previously available from Escalon Opthalmics, Inc. of Skillman, New Jersey placed in the superior conjunctival cul de sac had only 70% retention at two weeks. This implant is not typically visible to the patient. Therefore, the patient may not be aware of extrusion of the implant. There may be a period of time when no medication is administered potentially resulting in vision loss. Frequent visits to the doctor would be required to check the implant thus producing the same compliance problem that the implant is supposed to avoid.

An extended wear contact lens that releases drug is proposed as disclosed in Ciolino, et al., U.S. Pat. No. 8,414,912. This extended wear contact lens has to be regularly replaced, such as monthly. This can be difficult for many elderly patients and require monthly doctor visits, raising the same compliance issues noted above. Furthermore, the incidence of corneal ulcers, which can cause severe visual loss, is 10 to 15 times higher with extended wear contact lenses than with daily wear contact lenses.

A drug releasing implant that is placed on the surface of the eye has been proposed for example in Leahy, et al., U.S. Pat. No. 8,167,855. Another is a bimatoprost ring that fits in the fornices and rests atop the conjunctiva, extending 360 degrees about the cornea, which is available from Allergan of Dublin, Ireland. Such implants also have the problem of inadvertent extrusion or otherwise dislodgement, while remaining unrecognized by the patient, resulting in the disuniform release of the drug over the eye.

A drug releasing implant can be placed in the lacrimal drainage system, specifically the punctum and canaliculus, as disclosed in de Juan, Jr., et al., U.S. Pat. No. 7,998,497. An implant has been developed for the short term release of a corticosteroid. The lacrimal drainage system starts in the very medial upper and lower eyelids and is a series of ducts that drain tears into the nose. There are puncta on the medial upper and lower lids which drain into the upper and lower canaliculi, leading to the common canaliculus, then on into the lacrimal sac and down the nasolacrimal duct into the nose.

A drug releasing implant for placement into the punctum and canaliculus for longer term release of glaucoma medication has been proposed, as disclosed in Rapaki, et al., U.S. patent Ser. No. 10/434,009. The punctum which is the opening of the lacrimal drainage system to the ocular surface is small having a diameter of about 0.3 mm. Therefore, a very small surface area is available for release of the drug to the ocular surface. Repeated dilation of the punctum to place the implant is painful and makes extrusion more likely each time a new implant is placed. For example, lacrimal punctal plugs used to treat dry eye syndrome have a very high extrusion rate. Other intracanalicular plug systems include the Latanoprost Punctal Plug Delivery System (L-PPDS), and Evolute brand plug system by Mati Therapeutics, Inc. of Austin, Texas.

The Dextenza brand insert available from Ocular Therapeutix, of Bedford, Massachusetts is a FDA approved intracanalicular insert placed through the lower punctum. It delivers the corticosteroid dexamethasone to the ocular surface for up to 30 days. It is to be used after cataract surgery. The implant biodegrades and drains through the nasolacrimal duct after use.

Drug releasing implants have also been injected into the eye. Ozurdex brand implant available from Allergan of Irvine, California can be injected into the vitreous gel. It releases dexamethasone. The Ozurdex implant is a biodegradable implant used to treat diseases of the vitreous and retina such as diabetic retinopathy, uveitis, and macular edema.

Non-biodegradable drug releasing implants have been proposed for the vitreous cavity. Vitrasert brand implant available from Bausch & Lomb Incorporated of Irvine, California can be implanted for cytomegalovirus (CMV) retinitis. It releases ganciclovir for five to eight months. Retisert brand implant also available from Bausch & Lomb is a drug releasing implant that releases fluocinolone for 2.5 years for chronic noninfectious uveitis. Iluvien releases fluocinolone in the vitreous cavity. Surodex brand implant available from Oculex Pharmaceuticals of Sunnyvale, California is a metal implant that releases dexamethasone in the vitreous cavity for uveitis.

Subconjunctival injection of liposome loaded latanoprost and subconjunctival injection of microspheres of timolol maleate have been proposed. Drug releasing implants placed subconjunctivally have also been evaluated to treat glaucoma.

The surgical implantation of drug releasing implants in the anterior chamber also is under evaluation to treat glaucoma as disclosed in Tu, et al., U.S. Pat. No. 9,066,782. Such procedures require intraocular surgery and carry a risk of blinding endophthalmitis. Also drug releasing implants in the vitreous gel to treat retinal disease have been proposed, as disclosed for example in Nivaggioli, et al., U.S. Pat. No. 8,685,435. Many such implants may not be preferred due to difficulties associated with in implantation, irritation, and extraction.

The Drysta brand implant available from Allergan, of Irvine, California is a biodegradable FDA approved implant placed in the anterior chamber of the eye. It releases bimatoprost for the treatment of glaucoma. It lasts for a year or in some patients longer. There has been corneal endothelial cell loss associated with this implant.

None of the above described implants appear to release medication diffusely and completely over the ocular surface. Therefore the penetration of medication into the interior is irregular and suboptimal. They also appear to have the risks of infection, and extrusion. Some require major intraocular surgery. Many result in an inability of the patient to evaluate their presence, and are thus unaware of their extrusion. Others cause irritation of the eye and consequently have such a reduced size that the volume and duration of the medication released is reduced.

Therefore, there is a need for a drug delivery device and method which can supply medication to the eye in such a way which addresses one or more of the above problems.

### SUMMARY

The principal and secondary objects of the invention are to provide improved drug delivery device and method for supplying a drug to the eye.

These and other objects are achieved by a drug-releasing implant being implanted through the lacrimal caruncle into adjacent tissue.

In some embodiments the implant can be used to treat glaucoma, inflammation and other eye diseases.

In some embodiments the implant or some of its parts can be biodegradable or non-biodegradable.

In some embodiments there is provided a method for delivering a drug to orbital or periorbital tissues including the eye to treat a condition of a patient, said method comprises: forming a tract through tissues proximate to the lacrimal caruncle of a patient; placing an implant into said tract, selecting said implant to have: a body including a drug-carrying depot comprising: a drug selected to treat said condition; releasing said drug out of said implant and into said tissues; wherein said placing comprises: positioning said implant so that a proximal end of the implant is directly exposed to the lacrimal caruncle.

In some embodiments said forming comprises: further forming said tract through the lacrimal caruncle.

In some embodiments said forming comprises: further forming said tract through conjunctiva or other tissues surrounding the lacrimal caruncle, or the medial or inferomedial conjunctiva.

In some embodiments said forming comprises: extending said tract inferomedially from said lacrimal caruncle, surrounding conjunctiva, semilunar fold and adjacent tissues.

In some embodiments said placing further comprises: positioning said implant so that an exposed surface of the drug-carrying depot is directly exposed upon an outer surface of the lacrimal caruncle.

In some embodiments said placing further comprises: closing an opening in said lacrimal caruncle over a proximal end of said impact; whereby said implant is fully surrounded by tissue.

In some embodiments said forming further comprises: excising the lacrimal caruncle to form a tract through said lacrimal caruncle; and, dissecting said tract inferomedially from said lacrimal caruncle using a blunted instrument.

In some embodiments said method further comprises: allowing secretions emanating from said tissues to carry an amount of said drug out of said implant and onto the surface of the eye.

In some embodiments said method further comprises: said drug being a first drug; assessing whether said first drug has been in any way effective; and, recharging the implant with an amount of a second drug while said implant remains in vivo.

In some embodiments said first drug is different from said second drug.

In some embodiments said recharging comprises: injecting a flow of said second drug from a syringe into said depot.

In some embodiments said recharging comprises: said depot being a first depot; said first depot being removable from a remainder of said implant remaining in vivo; replacing said first depot with a second depot containing said amount of said second drug.

In some embodiments said method further comprises: allowing said implant to fully biodegrade within said tract.

In some embodiments said implant consists of one or more biodegradable and/or bioabsorbable materials.

In some embodiments said implant comprises one or more biodegradable and/or bioabsorbable materials.

In some embodiments said depot comprises one or more biodegradable and/or bioabsorbable materials, while a remainder of said implant comprises one or more non-biodegradable and/or non-bioabsorbable materials.

In some embodiments said method further comprises: retaining said implant in said tract during delivery of said drug onto said eye; wherein said retaining occurs in absence of fixating said implant to the tissues surrounding the lacrimal caruncle with a clamp or other device apart from said implant.

In some embodiments said method further comprises: retaining said implant in said tract during delivery of said drug onto said eye; wherein said retaining comprises providing said implant with a: an oblong axial trunk; a root located at a distal end of said trunk, said root having a radial diameter larger than an outside diameter of said trunk; and, a cap located at a proximal end of said trunk.

In some embodiments said placing comprises: locating said root at least 7.0 mm from a surface of said lacrimal caruncle.

In some embodiments said placing comprises: locating said root between 0.5 to 30 mm from a surface of said lacrimal caruncle.

In some embodiments said forming comprises: extending said tract inferomedially from said lacrimal caruncle; pushing a needle inferomedially through to the nasal cavity; and, wherein said placing comprises: locating said root adjacent to bone.

In some embodiments said retaining further comprises providing said implant with a plurality of ridges formed onto at least one of said trunk and said root.

In some embodiments said retaining further comprises providing said implant with at least one channel formed onto said root.

In some embodiments said retaining further comprises providing said implant with said cap having a radially expanded flange, and a layer of adhesive formed onto a distal surface of said flange bearing against an outer surface of said lacrimal caruncle.

In some embodiments said method further comprises: providing said implant with a window thereby exposing a surface of said depot to fluids in liquid communication with the eye.

In some embodiments said method further comprises: providing said implant with a dimensionally expanded button extending outwardly beyond said window; selecting a surface area for said button to adjust a flow rate of drug out of said depot.

In some embodiments said method further comprises: providing said implant with an opening in said body to a surface of said depot; wherein said opening is located apart from said window.

In some embodiments said method further comprises driving a flow of ambient body liquid through said depot between an inlet of said depot and an outlet of said depot, wherein said outlet is spaced apart from said inlet.

In some embodiments said method further comprises adjusting a diameter of a cavity containing said depot to control a rate of flow through said depot.

In some embodiments said method further comprises pumping a flow of said drug out of said implant.

In some embodiments said pumping comprises: providing said implant with a resiliently collapsible bladder containing a bulbous portion of said depot; collapsing a portion of said bladder thereby temporarily increasing a pressure of liquid in said depot.

In some embodiments said collapsing comprises: pushing a finger against the skin proximal to said bladder.

In some embodiments there is provided an implant for delivering a drug to orbital tissues including the eye to treat a condition of a patient, said implant comprises: a body; a drug-carrying depot comprising a drug; said implant being shaped and dimensioned to be inserted inferomedially through a tract in the lacrimal caruncle of the patient; whereby while the implant is located in the lacrimal caruncle of the patient, a surface of said drug-carrying depot is directly exposed to fluids in liquid communication with the eye.

In some embodiments said body further comprises: an oblong axial trunk; a root located at a distal end of said trunk, said root having a radial diameter larger than an outside diameter of said trunk; and, a cap located at a proximal end of said trunk.

In some embodiments said implant further comprises: a window through said cap exposing a proximal end of said depot.

In some embodiments said window is oversized creating a peripheral gap between said window and said proximal end.

In some embodiments said proximal end of said depot comprises: a dimensionally expanded button extending out beyond said window; wherein a surface area of said button is selected to adjust a flow rate of drug out of said depot.

In some embodiments said implant further comprises: a cavity located within said body, wherein said cavity is shaped and dimensioned to secure said depot therein.

In some embodiments said implant further comprises: said oblong axial trunk having an axial length of between 0.5 and 30 mm; said outside diameter of said trunk being between 0.01 and 7 mm; and, said radial diameter being between 0.1 and 12 mm.

In some embodiments said implant further comprises at least one prominence extending laterally from one of said trunk and root.

In some embodiments said prominence has a first axially perpendicular cross-sectional dimension greater than a second axially perpendicular cross-sectional dimension of said trunk.

In some embodiments said implant further comprises a plurality of axially spaced apart prominences extending radially from said body.

In some embodiments said implant further comprises a flange extending laterally at a proximal end of said body.

In some embodiments said flange comprises a distal flange surface oriented to rest against at least part of the tissue surrounding an opening of said tract when said implant is properly emplaced through said lacrimal caruncle.

In some embodiments said implant further comprises a layer of biocompatible adhesive located on said distal flange surface.

In some embodiments said implant further comprises a burl extending radially from a medial portion of said trunk.

In some embodiments said implant further comprises: a plurality of spaced apart windows through outer surfaces of said implant exposing surfaces of said depot.

In some embodiments said trunk comprises: a resiliently collapsible bladder containing a bulbous portion of said depot; whereby collapsing a portion of said bladder pumps a flow of drug out of said implant.

In some embodiments said body comprises a first non-biodegradable and/or non-bioabsorbable material and said depot comprises a first biodegradable and/or bioabsorbable material.

In some embodiments said body comprises a first biodegradable and/or bioabsorbable material and wherein said depot comprises a second biodegradable and/or bioabsorbable material.

In some embodiments said first biodegradable and/or bioabsorbable material is selected from the group consisting of: poly (lactic co glycolic acid) (PLGA), hydroxymethylcellulose, collagen, polydioxanone, E-Caprolactone-L-Lactide-copolymer, polycaprolactone (PCL)/PLGA, and polyethylene glycol (PEG).

In some embodiments said body further comprises biodegradeable particles selected from the group consisting of: colloidal particles, liposomes, microparticles, nanoparticles and nanospheres

In some embodiments said drug is dispersed in biodegradable polymeric matrix or membrane.

In some embodiments said drug comprises a therapeutic agent selected from the group consisting of: bimatoprost, latanoprost, latanaprostene bunod, netarsudil, tafluprost, travoprost, brinzolamide, betaxolol, carteolol, levobunolol, timolol, apraclonidine, brimonidine, ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, trifluridine, dexamethasone, triamcinolone, fluocinolone, and cyclosporine.

In some embodiments said drug comprises a therapeutic agent selected to treat dry eye syndrome.

In some embodiments said body is coated with a biodegradeable material.

In some embodiments said drug is conjugated to a biodegradeable material.

In some embodiments said implant establishes a flow of drug-containing fluid of at least 0.000001 milliliter per minute to said eye.

The content of the original claims is incorporated herein by reference as summarizing features in one or more exemplary embodiments.

### BRIEF DESCRIPTION OF THE DRAWINGS

FIG. 1 is a prior art diagrammatic partial cross-sectional illustration of the anatomical structure of the human orbit and lacrimal drainage system.

FIG. 2 is a diagrammatic partial cross-sectional illustration of the anatomical structure of a human orbit including a drug-releasing implant installed through the lacrimal caruncle.

FIG. 3 is a diagrammatic perspective view of a drug-releasing implant for installation through the lacrimal caruncle according to an exemplary embodiment of the invention.

FIG. 4 is a diagrammatic cross-sectional side view of the implant of FIG. 3.

FIG. 5 is a diagrammatic perspective view of a drug-carrying depot having an agglomeration of small particles containing a drug according to an alternate exemplary embodiment of the invention.

FIG. 6 is a diagrammatic side view of drug-carrying depot including small particles containing a drug carried within an enclosure according to an alternate exemplary embodiment of the invention.

FIG. 7 is a flow diagram of the primary steps for emplacing a drug-releasing implant through the lacrimal caruncle according to an exemplary embodiment of the invention.

FIG. 8 is a diagrammatic partial cross-sectional illustration of an excision step when implanting a drug-releasing implant through the lacrimal caruncle.

FIG. 9 is a diagrammatic partial cross-sectional illustration of a dissection step when implanting a drug-releasing implant through the lacrimal caruncle.

FIG. 10 is a diagrammatic partial cross-sectional illustration of a suturing step when implanting a drug-releasing implant through the lacrimal caruncle.

FIG. 11 is a diagrammatic partial cross-sectional illustration of a drug-releasing implant installed through the lacrimal caruncle and having a layer of tissue covering the proximal end.

FIG. 12 is a diagrammatic partial cross-sectional illustration of sutures closing an incision in the lacrimal caruncle to cover the proximal end of an installed drug-releasing implant.

FIG. 13 is a flow diagram of the primary steps for recharging a drug-releasing implant in vivo according to an exemplary embodiment of the invention.

FIG. 14 is a diagrammatic partial perspective and cross-sectional side view of an implanted lacrimal caruncle implant having its drug-carrying depot being replenished using a syringe.

FIG. 15 is a diagrammatic partial cross-sectional side view of an implanted lacrimal caruncle implant having its drug-carrying depot being replaced.

FIG. 16 is a diagrammatic partial cross-sectional side view of a lacrimal caruncle implant having its drug-carrying depot secured using an amount of adhesive.

FIG. 17 is a diagrammatic partial cross-sectional side view of an implanted lacrimal caruncle implant having a lyophilized drug-carrying replacement depot inserted into a cavity in the implant.

FIG. 18 is a diagrammatic partial cross-sectional side view of the implanted lacrimal caruncle implant of FIG. 17 after its drug-carrying depot has been saturated and expanded.

FIG. 19 is a diagrammatic partial cross-sectional side view of a lacrimal caruncle implant having a liquid permeable gate structure covering liquid access to the drug-carrying depot.

FIG. 20 is a diagrammatic partial cross-sectional side view of a lacrimal caruncle implant having a surface area expanding, axially extended button at the proximal end of the drug-carrying depot.

FIG. 21 is a diagrammatic partial cross-sectional side view of a lacrimal caruncle implant having a laterally expanded button at the proximal end of the drug-carrying depot.

FIG. 22 is a diagrammatic partial cross-sectional side view of a lacrimal caruncle implant having an expanded proximal cap carrying a drug-carrying depot and a liquid permeable gate structure.

FIG. 23 is a diagrammatic partial cross-sectional side view of a lacrimal caruncle implant having a drug-carrying depot having a proximal button extending to the radial periphery of the proximal cap, and a distal securing prong.

FIG. 24 is a diagrammatic cross-sectional side view of a lacrimal caruncle implant having surface area increasing ridges and grooves.

FIG. 25 is a diagrammatic cross-sectional side view of a lacrimal caruncle implant having ingrowth channels and proximal cap adhesive.

FIG. 26 is a diagrammatic cross-sectional side view of a lacrimal caruncle implant having a reduced lateral cross-section proximal cap.

FIG. 27 is a diagrammatic cross-sectional side view of a lacrimal caruncle implant having an extended proximal button structure having a reduced lateral cross-section.

FIG. 28 is a diagrammatic perspective view of a drug-releasing implant having a bone anchor for implantation through the lacrimal caruncle according to an alternate exemplary embodiment of the invention.

FIG. 29 is a flow diagram of the primary steps for emplacing a drug-releasing implant having a bone anchor through the lacrimal caruncle according to an alternate exemplary embodiment of the invention.

FIG. 30 is a diagrammatic partial cross-sectional illustration of the step of pushing a needle through the bone of the lateral nasal wall when implanting a drug-releasing implant having a bone anchor through the lacrimal caruncle.

FIG. 31 is a diagrammatic partial cross-sectional illustration of a tract widening step when implanting a drug-releasing implant having a bone anchor through the lacrimal caruncle.

FIG. 32 is a diagrammatic partial cross-sectional illustration of a tract dissection step when implanting a drug-releasing implant having a bone anchor through the lacrimal caruncle.

FIG. 33 is a diagrammatic partial cross-sectional illustration of an emplacement step when implanting a drug-releasing implant having a bone anchor through the lacrimal caruncle.

FIG. 34 is a diagrammatic cross-sectional side view of a drug-releasing implant having a bone anchor and without a middle root structure for implantation through the lacrimal caruncle according to an alternate exemplary embodiment of the invention.

FIG. 35 is a diagrammatic cross-sectional side view of a lacrimal caruncle implant having proximal and distal windows exposing opposite ends of the drug-carrying depot driving a flow of liquid therethrough.

FIG. 36 is a diagrammatic partial cross-sectional side view of a lacrimal caruncle implant having two medial windows exposing the drug-carrying depot to drive a flow of liquid therethrough.

FIG. 37 is a diagrammatic perspective view of a drug-releasing implant through the lacrimal caruncle which has a discharge tube for delivering drug to the eye according to an alternate exemplary embodiment of the invention.

FIG. 38 is a diagrammatic partial cross-sectional illustration of a drug-releasing implant installed through the lacrimal caruncle and having a discharge tube installed into the eyeball.

FIG. 39 is a diagrammatic perspective view of a manually pumpable drug-releasing lacrimal caruncle implant having a discharge tube for delivering drug to the eye according to an alternate exemplary embodiment of the invention.

FIG. 40 is a diagrammatic cross-sectional side view of the implant of FIG. 39.

FIG. 41 is a diagrammatic enlarged partial cross-sectional side view of the implant of FIG. 40 taken along circle 41-41.

FIG. 42 is a diagrammatic cross-sectional side view of the implant of FIG. 39 where the drug-carrying depot is compressed during a pumping action.

FIG. 43 is a diagrammatic enlarged partial cross-sectional side view of the implant of FIG. 42 taken along circle 43-43.

FIG. 44 is a diagrammatic partial cross-sectional illustration of an implanted manually pumpable drug-releasing lacrimal caruncle implant during a pumping action.

FIG. 45 is a flow diagram of the primary steps for emplacing and operating a manually pumpable drug-releasing implant according to an alternate exemplary embodiment of the invention.

### DESCRIPTION OF THE EXEMPLARY EMBODIMENTS

In this specification, the references to top, bottom, upward, downward, upper, lower, vertical, horizontal, sideways, lateral, back, front, proximal, distal, etc. can be used to provide a clear frame of reference for the various structures with respect to other structures typically while the patient is upright, and not treated as absolutes when the frame of reference is changed, such as when the device is inverted, disassembled, or the patient is lying down.

If used in this specification, the term “substantially” can be used because manufacturing imprecision and inaccuracies can lead to non-symmetricity and other inexactitudes in the shape, dimensioning and orientation of various structures. Further, use of “substantially” in connection with certain geometrical shapes and orientations, such as “parallel” and “perpendicular”, can be given as a guide to generally describe the function of various structures, and to allow for slight departures from exact mathematical geometrical shapes and orientations, while providing adequately similar function. Those skilled in the art will readily appreciate the degree to which a departure can be made from the mathematically exact geometrical references.

If used in this specification, the word “axial” is meant to refer to directions, movement, or forces acting substantially parallel with or along a respective axis, and not to refer to rotational nor radial nor angular directions, movement or forces, nor torsional forces.

In this specification the units “millimeter” or “millimeters” can be abbreviated “mm”, and “milligram” or “milligrams” can be abbreviated “mg”.

In this specification the term “drug” is meant to collectively include one or more medications, therapeutic agents, compounds, chemicals which are to be delivered to various structures of the eye and/or orbit by use of the implant to help treat some condition such as glaucoma or dry eye syndrome.

### Overview

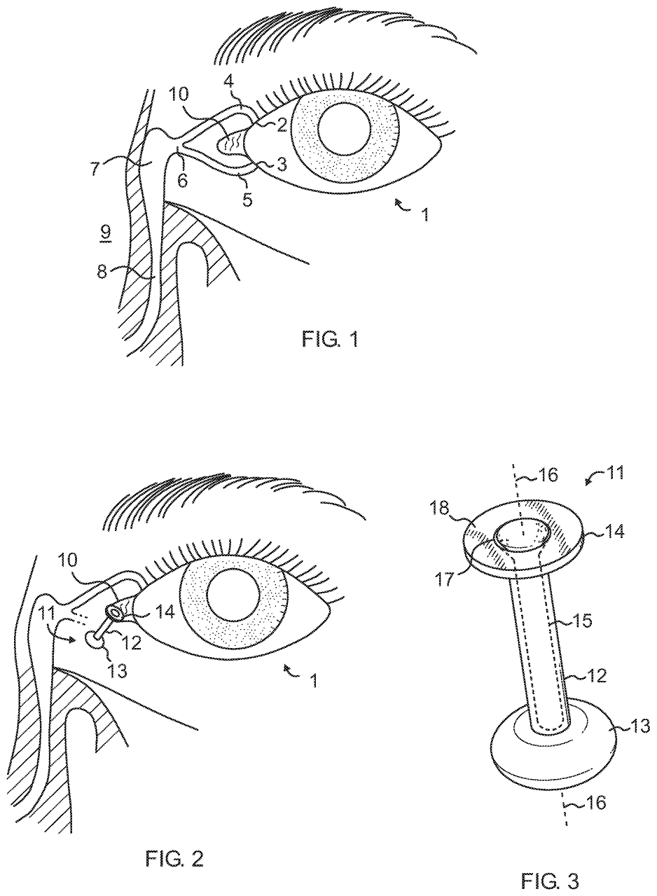

Referring now to the drawing, there is shown in FIG. 1 the medial anatomy of the orbit which includes a pair of small openings, namely the superior punctum 2 and inferior punctum 3, which are located on the medial upper and lower lids of the eye 1. These puncta lead to two small diameter delicate tubes, namely, the superior canaliculus 4 and the inferior canaliculus 5 which join together as a short common canaliculus 6 that enters into the larger lacrimal sac 7 leading to the nasolacrimal duct 8 and out an opening into the nasal cavity 9. These structures are part of the lacrimal system for draining tears. The lacrimal caruncle 10 is a fleshy nodule that lies in the medial canthus between the puncta.

Referring now to FIGS. 2-3, there is shown an exemplary embodiment of a drug-releasing implant 11 implanted through or proximal to the lacrimal caruncle 10 and into adjacent tissues. As will be described in greater detail below, the size, shape and location of the implant, once emplaced, allows it to be retained by the surrounding tissues with a very low or non-existent risk of being inadvertently extruded. In general, the implant can have an elongated body including a middle trunk portion 12, a distal anchor or root portion 13 that helps anchor the implant, and a proximal cap portion 14 which can rest external to the conjunctiva of the lacrimal caruncle. The trunk, root, and cap can be made from a unitary piece of biocompatible material.

A drug-carrying region or depot 15 can be loaded within a cavity extending through the cap 14 and into trunk 12, as shown, or in other parts of the implant body, as will be described below. A flow of drug can exit the depot through a drug-releasing window 17 on the exposed proximal surface 18 of the cap. The window exposes a proximal surface portion of the depot. In this way a flow of drug can be established by ambient bodily liquids eluting the drug out of the depot. Thus, the depot can be exposed to fluids which are in liquid communication with the eye and thus can provide drug treatment to the eye over an extended period.

Ambient bodily fluids, primarily tears, can contact the exposed implant encouraging the release of a flow of the drug out of the implant and lead to sustained diffusion of the drug into the tears. Once this flow is established, drug-carrying tears can be continuously supplied to the surface of the eye. The drug which is integrated into the tears completely and constantly coats the surface of the eye resulting in maximal penetration into the eye and maximal clinical effect. It is expected that a properly placed implant can release between about 0.0001 microgram and 1 milligram of drug per hour to the outer surface of the eye. In other words, the drug carried by the implant can be released by the implant over a typical designated period of time. The timed release can occur in absence of any intervention by the physician.

Because the lacrimal caruncle is less sensitive to foreign body intrusion, patients will tolerate the presence of the implant to a greater degree. In this way the implant can be made larger, more rugged, and more ruggedly emplaced. Being larger, the lacrimal caruncle implant can have a larger drug-releasing surface thereby providing greater sustainability to the drug flow, and avoiding clogging which can occur with smaller implants. Also, a greater reservoir of drug can be carried by the depot loaded into the implant, reducing the frequency of office visits to renew the operation of the implant.

### Shape

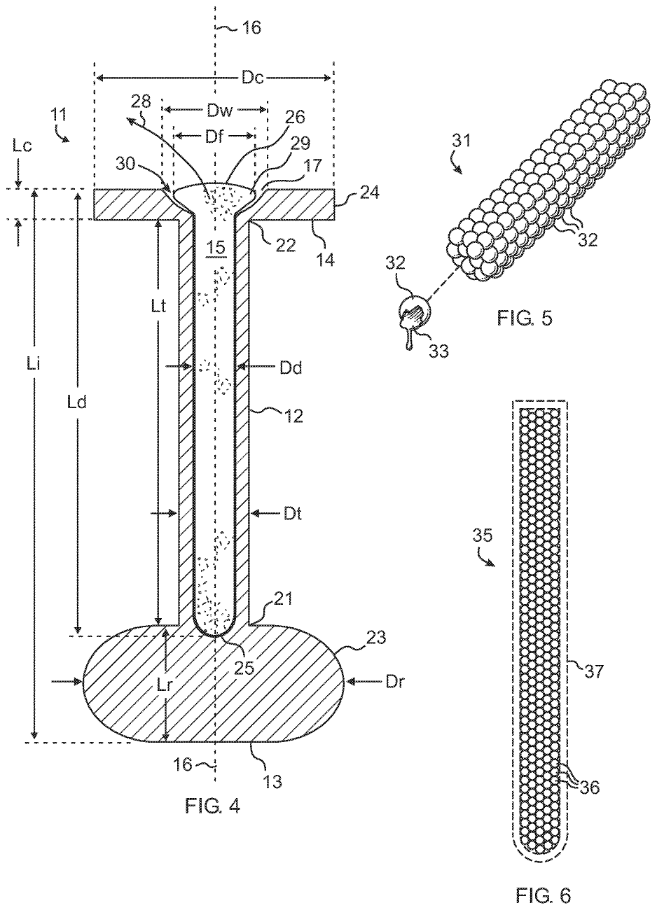

Referring now to FIG. 4, in this embodiment, as stated above, the implant 11 can have elongated body including a middle trunk portion 12, a distal anchor or root portion 13, and a proximal cap portion 14. The body can be elongated along and substantially symmetric about a central longitudinal elongation axis 16. The trunk can be substantially cylindrical in shape having an axial length Lt and a cross-sectional diameter Dt. The trunk can terminate at a distal end 21 connected to a radially enlarged root 13 having a substantially flattened oblate spheroid shape having an axial length Lr and a rounded peripheral edge 23 having a maximum diameter Dr, taken substantially perpendicular to the elongation axis, which is significantly larger than the diameter Dt of the trunk. The trunk can terminate at a proximal end 22 in an enlarged cap 14 having a substantially disk-shaped structure having an axial length Le and a substantially cylindrical peripheral edge 24 having a diameter De significantly larger than the trunk diameter in order to form a proximal flange structure. Thus, the overall axial length Li of the implant body can be defined by the equation Li=Le+Lt+Lr.

The implant 11 can include a drug-carrying depot 15 loaded into a cavity in the implant. The depot can have a substantially cylindrical body elongated along the central axis 16 of the implant, and can have an axial length Ld extending between a proximal end 26 and a distal end 25, and a cross-sectional diameter Dd. The axial length of the depot can extend beyond the axial length of the trunk as shown, or can extend the exact length of the trunk, or can be shorter than the length of the trunk. In this embodiment the depot dimensions can be selected so that the depot is partially enclosed within the implant body, having only its proximal end exposed through a proximal window 17 of the implant body from which drug can flow 28 out of the depot. The depot can have a substantially conical proximal flare 29 allowing the proximal end to have a larger exposed circular surface area to enhance the flow and reduce the risk of debris clogging the window. In other words, the flare can extend laterally having a diameter Df beyond the cross-sectional diameter Dd of the depot in its middle portion.

For the most part, the shape and dimensions of the depot 15 can be selected to closely match the shape and dimensions of the cavity in the implant 11 in order to retain the depot within the cavity and maximize the volume of the depot. However, the window 17 of the cavity can be oversized to allow a peripheral gap 30 between the window and proximal end 26 of the depot to enhance the flow of liquid around the exposed surfaces of the depot to help the dispersal of drug from the depot to the eye.

In some embodiments the cap is intended to be located external to the lacrimal caruncle and thus rest against the conjunctival surfaces. In this way the cap flange can help avoid ingrowth of tissue which could cover the drug-releasing window to the depot. In other embodiments an amount of tissue covering the proximal end of the implant, including the covering over of the cap entirely, can be encouraged.

For a typical human implantation the implant body can have the following exemplary range of dimensions:

| Dimension: | Minimum (mm): | Typical (mm): | Maximum (mm): |
|---|---|---|---|
| Lc | 0.01 | 1 | 3 |
| Lt | 0.5 | 7 | 30 |
| Lr | 0.01 | 1 | 5 |
| Dc | 0.1 | 4 | 10 |
| Dt | 0.01 | 2.5 | 7 |
| Dr | 0.1 | 4 | 12 |

For implants made of resilient materials the above dimensions are given for the implant at rest. It shall be understood that other shapes having different dimensioning on different segments of the implant body can be used depending on manufacturing, surgical implantation, drug flow rates, drug types and other concerns.

Although the drug-carrying lacrimal caruncle implant can be of a variety of shapes, the above-described shape and dimensioning are selected so that it can be simply manufactured, surgically installed, and if necessary surgically removed. The above-described shape allows the implant to be completely lodged within the tissues extending inferomedially from the lacrimal caruncle or surrounding conjunctiva and nearby tissues and to not interfere with neighboring tissues. The shape can also be selected to reduce inflammation, irritation and pain. Further, it allows the implant to be emplaced in absence of fixating structures attaching the implant to the surrounding tissues such as with a clamp or other structure apart from the implant itself. This does not include the temporary sutures used to heal the tissues surrounding the implant immediately after emplacement in some embodiments.

### Materials

The drug-releasing implant can be made from biodegradable materials which are typically eventually absorbed by the body, in which case the implant need not be removed. In this way, apart from implantation, treatment can occur in absence of further physician intervention, thereby allowing the implant to biodegrade. In other words, the treatment can occur in absence of any surgical removal of the implant from the tract. Alternately, the implant can be made from non-biodegradable materials, in which case the implant can eventually be removed from the lacrimal caruncle. Regardless of whether the implant is made from biodegradable or non-biodegradable materials, the implant can be recharged in vivo with an additional amount of drug as will be described in greater detail below. For example, the implant can have a non-biodegradable body which carries a biodegradable drug-carrying depot. Once the depot degrades it can be replaced with a new depot as will be described in greater detail below.

The biodegradable drug-releasing implant for implantation in the lacrimal caruncle can be made of a variety of materials or their combination. Those materials include biodegradable poly (lactic co glycolic acid) (PLGA), hydroxymethylcellulose, collagen, polydioxanone, E-Caprolactone-L-Lactide-copolymer, polycaprolactone (PCL)/PLGA, polyethylene glycol (PEG), and other biodegradable and/or bioabsorbable materials known to be compatible with the body.

Further, the biodegradable drug-releasing implant can be coated with PLGA, hydroxymethylcellulose, polycaprolactone (PCL)/PLGA and combinations thereof.

Further, the drug can be conjugated to PLGA, hydroxymethylcellulose, PCL/PGLA or combinations thereof.

The non-biodegradable drug-releasing implant for implantation in the lacrimal caruncle, including the root, trunk, cap, and depot can be made from a variety of biocompatible materials or their combination. Those materials can include silicone, silicone combined with other polymers, poly(methyl methacrylate) (PMMA), cross-linked hydrogels, NN-dimethylacrylamide, methacrylic acid, poly(ethylene-co-vinyl acetate), microspheres, liposomes, polyvinyl alcohol (PVA), ethylene vinyl acetate, polytetrafluoroethylene (PTFE), metallic non-ferrous materials such as titanium, glass, teflon and other materials known to be compatible with the body at least for a limited time.

It shall be understood that the drug-carrying depot 15 as a part of the implant can be made from a biocompatible, liquid permeable materials that are partially or fully biodegradable, bioabsorbable, non-biodegradable, or non-bioabsorbable. For example the depot may comprise or consist of biodegradable colloidal particles such as liposomes, microparticles, and nanoparticles, or other biodegradable and or bioabsorbable material known to those experienced in the art. Biodegradable microparticles containing medication may be composed of PLGA or PLA or other biodegradable material. Nanoparticles containing medication may be incorporated into biodegradable material such as polyethylene glycol (PEG) or PEG coated nanoparticles. The depot can be formed by a block of such particles dispersed in a biodegradable polymeric matrix or membrane, for example.

Those skilled in the development of such drug delivering implants will appreciate that the implant can be made from materials which are soft or hard, rigid or flexible depending on the application. Further the entire implant can be made to be biodegradable.

As shown in FIG. 5, for example, the depot body 31 can be an agglomeration of small particles 32 each containing an amount of drug 33 which can be dispensed from the implant as the particle coatings dissolve or are otherwise are broken. The particles can be agglomerated to one another by an amount of interconnecting biocompatible glue of a type known in the art such as adhesive such as Surgiseal brand 2-octyl cyanoacrylate adhesive, commercially available from Adhezion Biomedical, LLC of Wyomissing, Pennsylvania.

As shown in FIG. 6 the depot body 35 can be a grouping of small particles 36 each containing an amount of drug which can be dispensed from the implant as the particle coatings dissolve. The particles can be carried within a biocompatible, liquid permeable enclosure 37 which itself can be biodegradable, bioabsorbable, non-biodegradable, or non-bioabsorbable.

### Medications

The drug-releasing implant, whether partially or fully biodegradable, bioabsorbable, non-biodegradable, or non-bioabsorbable can include a number of medications, therapeutics agents, compounds, or chemicals, either alone or in combination for delivery to the structures of the eye and/or orbit. Any of the above, alone or in combination, are referred to as a drug in this specification.

For example, the drug can include one or more glaucoma medications such as bimatoprost, latanoprost, latanaprostene bunod, tafluprost, travoprost, brinzolamide, betaxolol, carteolol, levobunolol, timolol, apraclonidine, brimonidine, rocklatan, and netarsudil.

The drug can include one or more anti-infective medications such as ganciclovir, acyclovir, famcyclovir, trifluridine, besifloxacin, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, and trifluridine.

The drug can include one or more anti-inflammatory medications such as dexamethasone, triamcinolone, fluocinolone, cyclosporine, lifitegrast, and ketorolac.

In addition, the drug can include one or more parasympathomimetic medications such as pilocarpine or artificial tears.

The drug can include one or more androgens such as testosterone, dihydrotestosterone, methyltestosterone, testosterone cypionate, oxandrolone, danazol or other androgens.

The drug can include one or more lubricants, artificial tears, and gels.

### Surgical Method

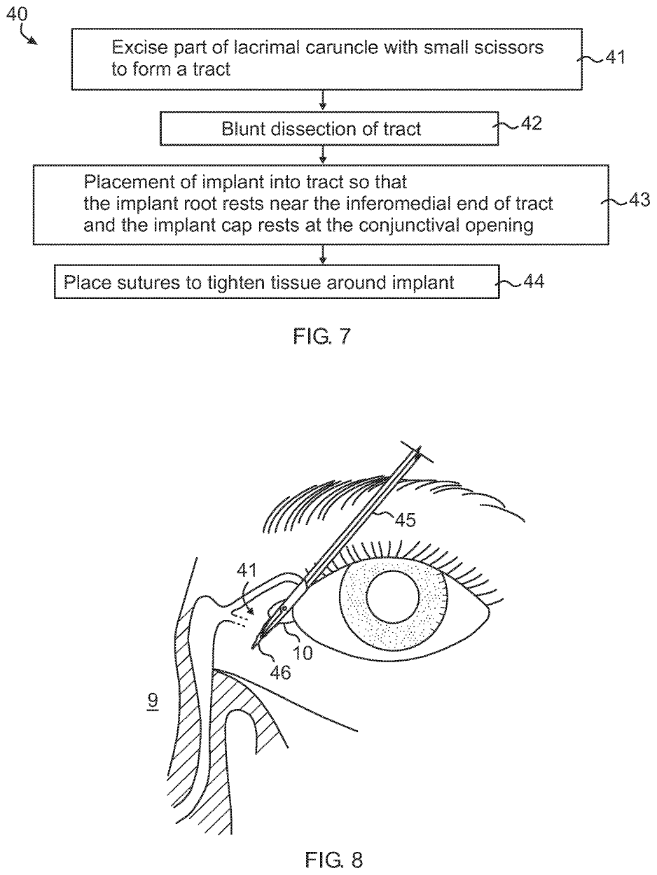

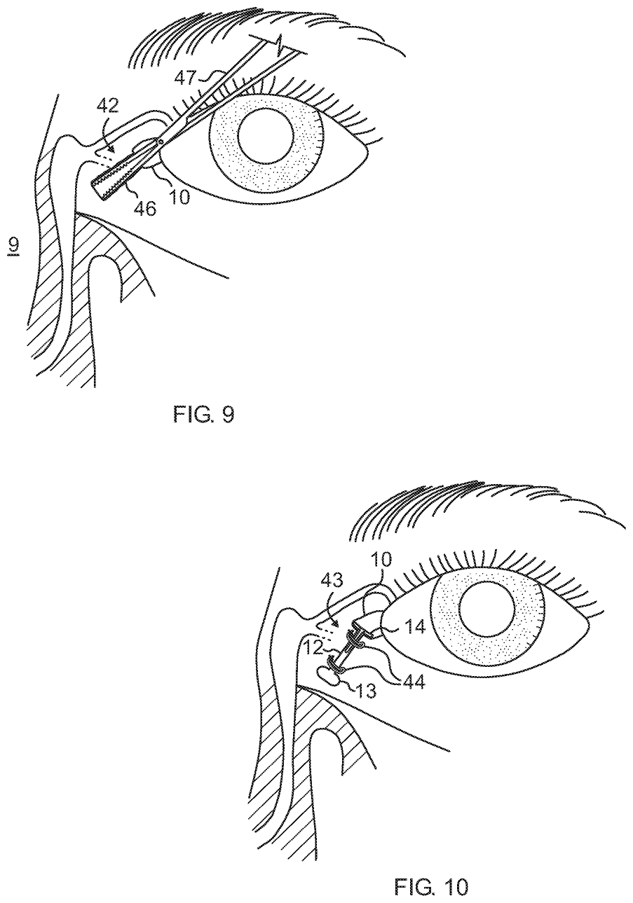

Referring now to FIGS. 7-10, there is described an exemplary embodiment of a surgical method 40 for installing a drug-releasing lacrimal caruncle implant. Part of the lacrimal caruncle 10, typically the inferior half, can excised 41 with small scissors 45 to form a tract 46 having a small opening. In some cases the entrance may be through other medial canthal conjunctiva such as the semilunar fold or surrounding areas that are proximal to the lacrimal caruncle. Blunt dissection 42 of the tract 46 with a hemostat 47, scissors, or other instrument can then be carried out aiming inferomedially toward the nasal cavity 9. This may be carried as deep as the bone of the lacrimal fossa. The implant can then be placed 43 within the tract so that the root 13 is deep within the tract and the cap 14 of the implant rests against the conjunctival opening on an outer surface of the lacrimal caruncle. Typically, and depending on the dimensioning of the implant, this will result in the root being located between 0.5 and 30 mm, and usually at least 7.0 mm from the outer surface of the lacrimal caruncle. Sutures 44 can then be placed to bring the surrounding tissues tight up against the trunk 12 of the implant. This will prevent extrusion as the surrounding tissues will form a tight scar around the trunk.

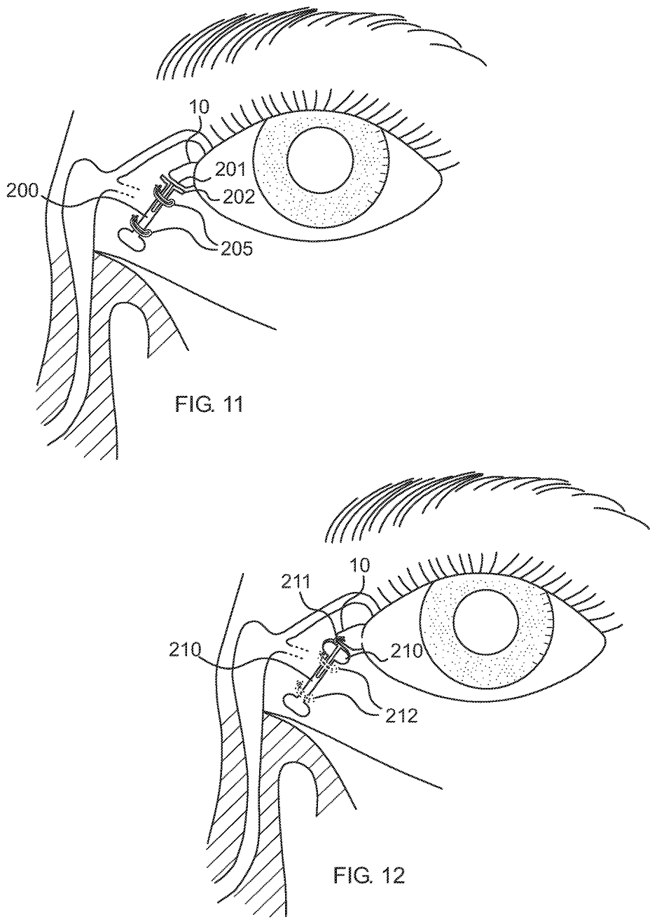

As shown in FIG. 11, the surgical method can be adapted to allow a layer 201 of conjunctival tissue to exist over the proximal end 202 of the implant 200. In this way, the implant is intended to be fully enclosed within the tract formed inferomedially from the lacrimal caruncle 10 so that the layer of conjunctiva tissue covers over the proximal end of the implant. Sutures 205 can help keep the implant properly emplaced until the layer overgrows the opening through the lacrimal caruncle.

As shown in FIG. 12, the surgical method can be further adapted to provide additional suturing 211 to close the opening through the caruncle 10 in order to enclose the implant 210 within the created inferomedial tract. Suturing 212 on the trunk can then be optional depending size of the tract and other factors. Because the implant is substantially fully enclosed within the tract, the proximal flange 213 can be made to have a more rounded shape to improve comfort, or be eliminated altogether.

### Recharging

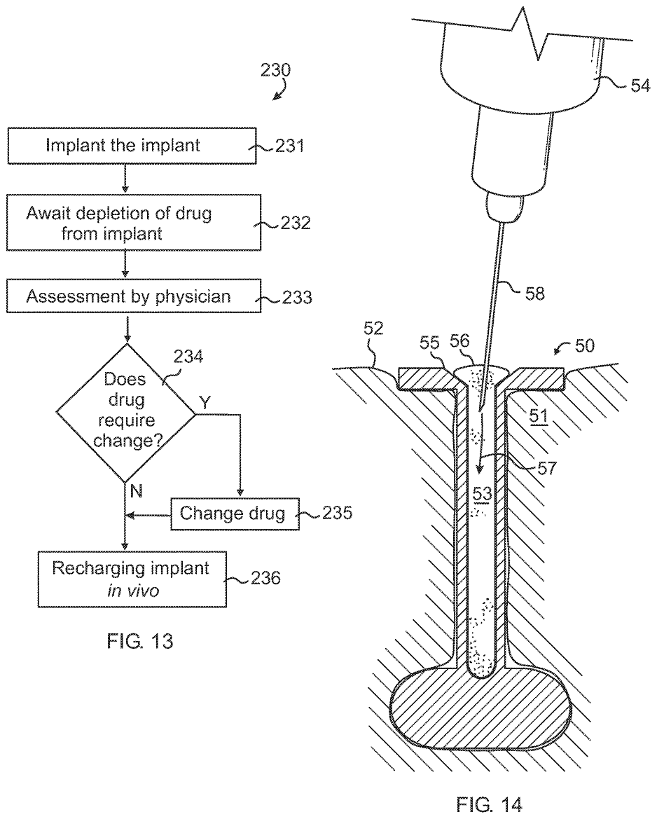

Referring now to FIG. 13, there is described an exemplary embodiment of a method 230 for the in vivo recharging a drug-releasing lacrimal caruncle implant that has become drug depleted. The steps can include the surgical installation 231 of the implant through the lacrimal caruncle as described above. The implant then dispenses its drug over time. After an amount of time has passed 232 the implant can become drug depleted. At this time the physician can assess 233 whether the drug treatment has been effective and determine what course to take. For example, the physician may determine 234 whether a change 235 in medication is indicated. Once that determination is made, the implant can be recharged in vivo 236 with a new amount of either the same drug used previously or a different drug. Recharging of the implant can occur in a number of ways.

As shown in FIG. 14, for example, a drug-releasing lacrimal caruncle implant 50 has been installed into tissues 51 through the surface 52 of the lacrimal caruncle. This embodiment shows that a drug-carrying depot 53 which has become drug-depleted can be recharged in vivo using a syringe 54 having a needle 58 penetrating through the window 55 in the proximal cap of the implant and into the exposed proximal end 56 of the depot. In this way the syringe can inject a flow 57 of additional or alternate drug depending on the needs of the patient after the effectiveness of the previous round of treatment has been assessed. In this way the implant can be recharged with additional or alternate medication in absence of the removal of the implant from the body. It shall be noted that recharging the depot using this injection method can occur when the proximal cap of the implant is covered with tissue as shown in FIGS. 11-12. Further, the depot having a widened surface area as shown in FIG. 4 can allow the surgeon to more easily “hit the target” with the syringe during a recharge procedure.

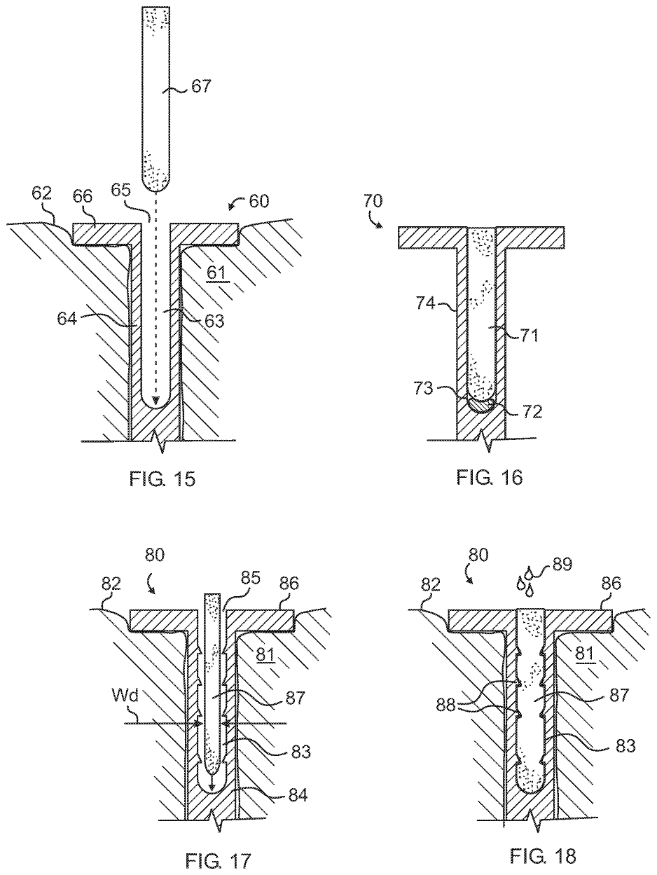

Referring now to FIG. 15, there is shown an exemplary embodiment of a drug-releasing lacrimal caruncle implant 60 that has been installed into tissues 61 through the surface 62 of the lacrimal caruncle. This embodiment shows that the original drug-carrying depot has become drug-depleted and has either biodegraded or been removed from a cavity 63 extending axially within the trunk 64 of the implant to an exposed opening 65 in the cap 66. The implant can be recharged in vivo by installing into the cavity a second, fresh depot 67 containing the necessary drug. As with the previous embodiment the physician can assess at the time of recharging whether a change in the drug being administered should occur.

In FIG. 16 there is shown an exemplary embodiment of a drug-releasing lacrimal caruncle implant 70 wherein the removable depot 71 can be secured within the cavity 73 of the trunk 74 by a patch 72 of biocompatible adhesive such as Surgiseal brand 2-octyl cyanoacrylate adhesive, commercially available from Adhezion Biomedical, LLC of Wyomissing, Pennsylvania. The patch of adhesive will help the depot remain in place in the cavity until removed or replaced at a later time.

Referring now to FIGS. 17-18, there is shown an exemplary embodiment of a drug-releasing lacrimal caruncle implant 80 that has been installed into tissues 81 through the surface 82 of the lacrimal caruncle. This embodiment shows that the original drug-carrying depot has become drug-depleted and has either biodegraded or been removed from a cavity 83 extending axially within the trunk 84 of the implant to an exposed opening 85 in the cap 86. The implant can be recharged in vivo by installing a second, fresh depot 87 containing the necessary drug. The physician can assess at the time of recharging whether a change in the drug being administered should occur.

This embodiment shows that the replacement depot 87 can be made from a lyophilized hydrophilic material containing an amount of drug. In a dried and compressed state as shown in FIG. 17, the replacement depot can have a cross-sectional dimension Wd much smaller than the opening 85 to the cavity 83, and thus can be easily inserted into the cavity while the implant remains in vivo.

As shown in FIG. 18, once the replacement depot 87 has been fully inserted into the cavity 83, an amount of liquid 88 such as tears or other bodily liquids can saturate the depot and allow it to expand to fill the cavity. Barbs 89 on the inner surface of the cavity can contact the expanded depot to help prevent inadvertent withdrawal of the depot from the cavity.

### ALTERNATE EMBODIMENTS

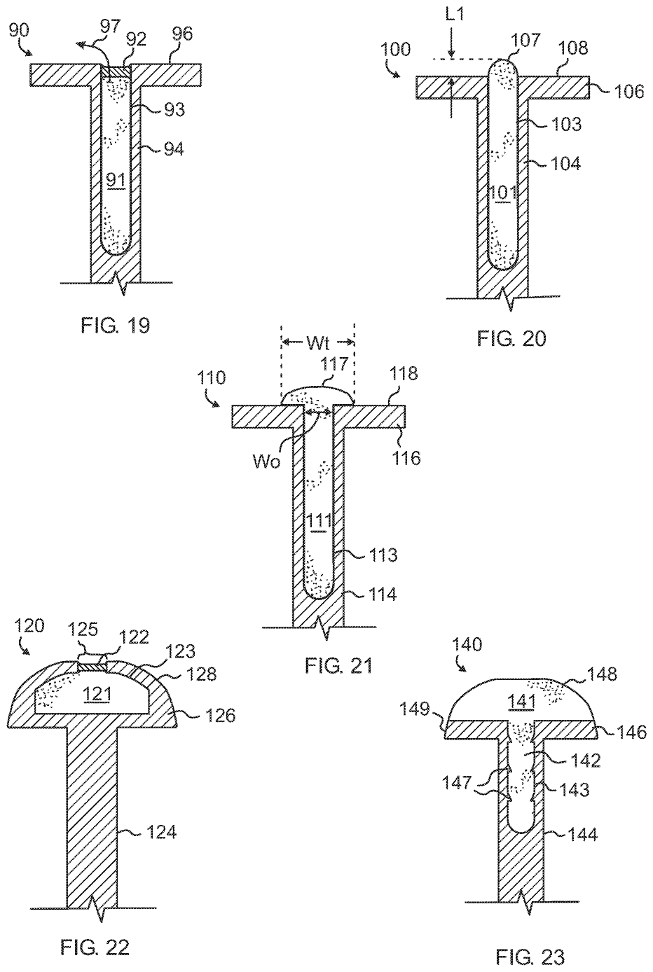

Referring now to FIG. 19, there is shown an exemplary embodiment of a drug-releasing lacrimal caruncle implant 90. This embodiment shows that the drug-releasing depot 91 can be contained within a cavity 93 extending axially within the trunk 94 of the implant. This embodiment shows that the opening at the proximal end of the cavity can be closed by a gate structure 92 made from a liquid permeable, biocompatible material such as liquid permeable silicone. The dimensions of the gate, such as its axial thickness can be selected to regulate the flow 97 of drug being released from the depot. The gate can be made from a resilient material which allows the depot to be recharged using a syringe as shown in the embodiment of FIG. 14.

Referring now to FIG. 20, there is shown an exemplary embodiment of a drug-releasing lacrimal caruncle implant 100 that can include a drug-releasing depot 101 carried within a cavity 103 extending axially within the trunk 104 of the implant. This embodiment shows that a proximal portion of the depot can extend outwardly from the window an axially proximal distance L1 beyond the substantially planar proximal surface 108 of the flange 106 forming the proximal cap of the implant. In this way the exposed surface area of the depot may be increased to form a button 107 in order to allow a greater flow of drug out of the depot. For implantations allowing a layer of conjunctival tissue to cover the proximal cap of the implant as shown in FIGS. 11-12, the dimensions of the button can be selected to allow the button to remain beneath the layer or to poke through the layer.

Referring now to FIG. 21, there is shown an exemplary embodiment of a drug-releasing lacrimal caruncle implant 110 that can include a drug-releasing depot 111 carried within a cavity 113 extending axially within the trunk 114 of the implant. Similar to the embodiment of FIG. 20, this embodiment shows that a proximal button portion 117 of the depot can extend outwardly from the window, axially and proximally beyond the substantially planar proximal surface 118 of the flange 116 forming the proximal cap of the implant. It also shows that the button portion can extend laterally so that is has a width dimension Wt that is larger than the width dimension Wo provided by the proximal opening to the cavity. In this way the exposed surface area of the depot may be selected in order to adjust the flow rate of drug out of the depot. For example, a button surface area of between about 0.1 and 10 mm² can be selected resulting in a flux of sustained drug release of approximately 0.000001 to 100 milliliter/hour.

Referring now to FIG. 22, there is shown an exemplary embodiment of a drug-releasing lacrimal caruncle implant 120 that can include a drug-releasing depot 121 carried within a cavity 123 formed into the interior of an expended dimension cap 126 of the implant. The cap can have a convex, rounded outer proximal surface 128 for comfort and to avoid regions which could trap debris. An axially central proximal opening 125 in the exposed top of the cap can lead to the depot. Similar to the embodiment of FIG. 19, the opening can be closed by a gate structure 122 made from a liquid permeable, biocompatible material. The dimensions of the gate can be selected to regulate the flow of drug therethrough. The gate can be made from a resilient material which allows the depot to be recharged using a syringe.

Referring now to FIG. 23, there is shown an exemplary embodiment of a drug-releasing lacrimal caruncle implant 140 that can include a drug-releasing depot 141 formed to rest atop the proximal cap 146 of the implant. The depot can have a convex, rounded outer proximal surface button 148 which can be commensurate with the lateral or peripheral lateral or radial edge 149 of the cap for comfort and to avoid regions which could trap debris. The depot can have a distally extending prong 142 which can engage a cavity 143 extending axially within the trunk 144 of the implant. Barbs 147 on the inner surface of the cavity can contact the prong to help prevent inadvertent withdrawal of the prong from the cavity.

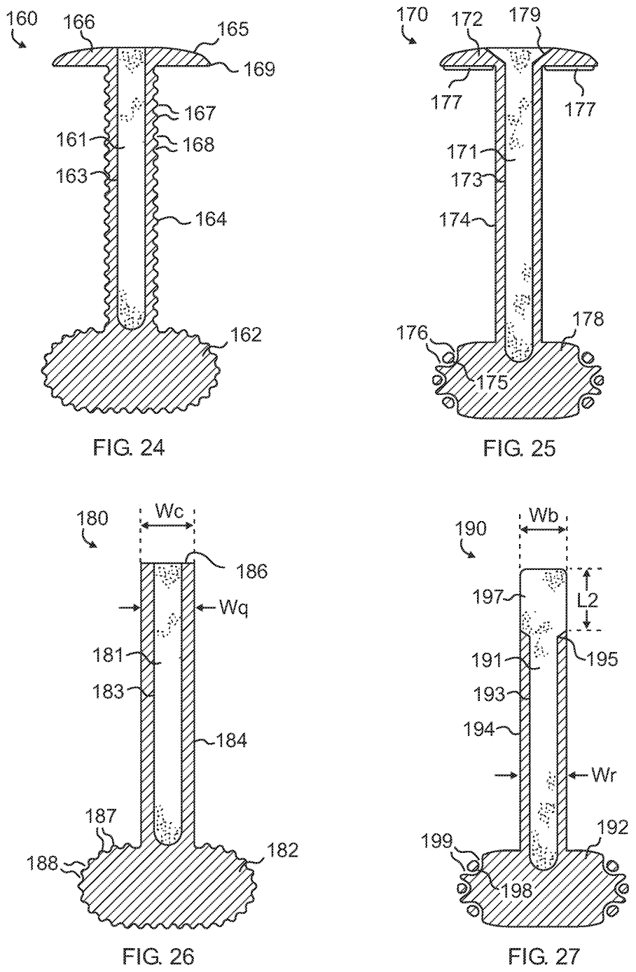

Referring now to FIG. 24, there is shown an exemplary embodiment of a drug-releasing lacrimal caruncle implant 160 that can include a drug-releasing depot 161 carried within a cavity 163 extending axially within the trunk 164 of the implant. This embodiment shows that the implant can include ridges or other surface features such as one or more prominences 167 which can be spaced apart by grooves 168 extending radially inwardly from the radially outward-most part of the prominences. The surface features can increase the surface area of the implant to allow greater exposure to the surrounding tissues, and to create surfaces that increase friction to axial movement thereby helping prevent axial migration of the implant after emplacement, and thereby further securing the position of the implant when it extends inferomedially from the lacrimal caruncle. A roughened surface having various protrusions can aid in the adherence of scar tissue to further prevent extrusion. This embodiment shows that the surface of the prominences and grooves can transition smoothly to avoid nooks or other structures which would tend to trap debris which could lead to unwanted bacterial propagation.

Referring now to FIG. 25, there is shown an exemplary embodiment of a drug-releasing lacrimal caruncle implant 170 that can include a drug-releasing depot 171 carried within a cavity 173 extending axially within the trunk 174 of the implant. This embodiment shows that the implant can include at least one channel 175 extending through the root 178 of the implant to at least a pair of inlets 176. One or more channels can be formed in the root or other parts of the implant body. Each channel can provide a path for the ingrowth of tissue to better secure the implant thereby helping prevent axial migration of the implant after emplacement inferomedially through the lacrimal caruncle. Further, the channel can act as a rugged attachment point for sutures. The surfaces of the implant bordering the channel and inlets can transition smoothly to avoid nooks or other structures which would tend to trap debris which could lead to unwanted bacterial propagation. An optional layer of biocompatible adhesive 177 can be present on the distal surface of the flange 172 that faces the conjunctiva in order to further help prevent extrusion. The flange can also act as a readily exposed grasping point for removing the implant. As with many embodiments of the implant, the depot 171 can have a substantially conical proximal flare 179 allowing the proximal end to have a larger exposed circular surface area to enhance the flow and reduce the risk of debris clogging the window.

Referring now to FIG. 26, there is shown an exemplary embodiment of a drug-releasing lacrimal caruncle implant 180 that can include a drug-releasing depot 181 carried within a cavity 183 extending axially within the trunk 184 of the implant. This embodiment shows that the implant can include a proximal cap 186 at the proximal end of the implant that has a reduced lateral cross-section over that of some other embodiments for improved comfort. The cap can have a greatest width dimension We which is less than or equal to the width dimension Wq of the trunk. In this embodiment it is determined that the root structure 182 can provide adequate resistance to unintended extrusion by, for example, providing surface texturing such spaced apart ridges 187 and grooves 188 to enhance the anchoring properties of the root. It shall be noted that the reduced size proximal cap can be used in implantations where the proximal end of the implant is exposed through the lacrimal caruncle or covered with tissue.

Referring now to FIG. 27, there is shown an exemplary embodiment of a drug-releasing lacrimal caruncle implant 190 that can include a drug-releasing depot 191 carried within a cavity 193 extending axially within the trunk 194 of the implant. This embodiment shows that a proximal portion of the depot can form a button 197 extending outwardly from a window 195 at the proximal end of the cavity. The button can extend an axially proximal distance L2 beyond the window providing an enlarged exposed surface area to increase the flow of drug out of the implant. The button can have a greatest width dimension Wb which is greater than, less than or equal to the width dimension Wr of the trunk. In this embodiment it is determined that the root structure 192 of the implant can provide adequate resistance to unintended extrusion by, for example, providing at least one channel 198 extending through the root 192 to at least a pair of inlets 199 allowing for tissue ingrowth. It shall be noted that the extended proximal button structure of the depot can be used in implantations where the proximal end of the implant is exposed through the lacrimal caruncle or covered with tissue.

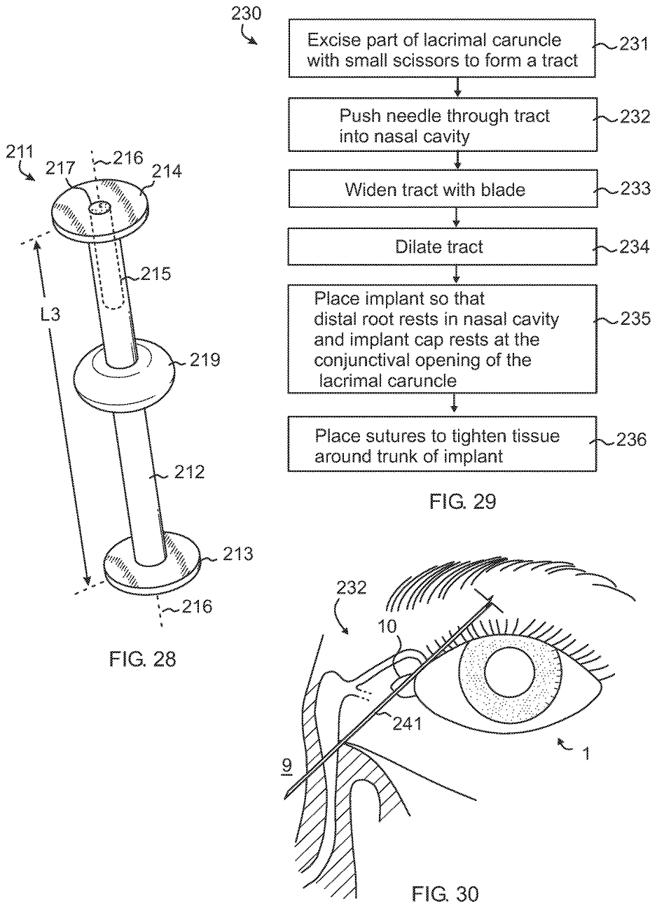

Referring now to FIG. 28 there is described an exemplary embodiment of a drug-releasing lacrimal caruncle implant 211 having an axially extended trunk portion 212 that allows the implant to extend through the bone of the lateral nasal wall into the nasal cavity and allowing a distal root 213 to bear against the medially facing surface of the nasal wall thereby enhancing resistance to extrusion and further securing the position of the implant when it extends inferomedially from the lacrimal caruncle. As with prior embodiments the implant can include a drug-releasing depot 215 carried within a cavity in the trunk and exposed through a window 217 in a proximal cap 214 in order to a flow of drug to exit the depot. The distal root 213 can be formed by a laterally or radially projecting flange-type structure that can collapse against the trunk during insertion and resiliently expand laterally in the nasal cavity. An optional radially enlarged burl structure 219 can be formed on an axially medial portion of the trunk to further enhance resistance to extrusion or even migration toward the nasal cavity. Indeed, the enlarged burl structure can be applied to other embodiments disclosed herein including those that do not anchor to bone.

In order for the root 213 to locate in the nasal cavity while the proximal cap 214 is located near the lacrimal caruncle the implant 211 can have an axial length L3 of between 3 and 50 mm, and for most adult patients between about 10 and 25 mm.

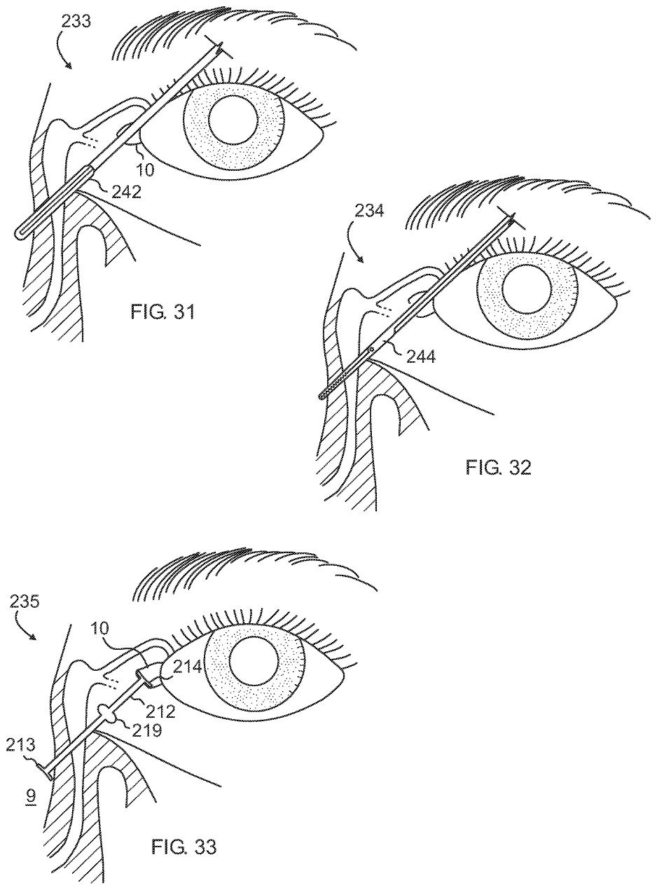

Referring now to FIGS. 29-33 there is described an exemplary embodiment of a method 230 for installing the bone-secured lacrimal caruncle implant of FIG. 28. The inferior half of lacrimal caruncle 10 can be excised 231 with scissors to form an opening. A tract can be formed 232 by pushing large gauge needle 241 through the opening of the inferior lacrimal caruncle inferiorly and medially through soft tissue and the lateral nasal wall including bone of lateral nasal wall into the nasal cavity 9. A plate can be used to protect the nasal septum and is placed prior to pushing the needle. The needle can then be withdrawn. The tract can be widened 233 by pushing a #69 Beaver brand blade 242 through the inferior lacrimal caruncle medially and inferiorly through the tract into the nose. The blade can then be withdrawn. The tract can be dilated 234 by using a mosquito hemostat 244. The implant can then be placed 235 in the tract by pushing from the opening in the lacrimal caruncle, through the dilated tract until the root 213 comes to rest in the nasal cavity 9 adjacent to the bone, either against it or close to it, and the proximal cap 214 can rest at the conjunctival opening of the lacrimal caruncle.

It shall be noted that the bone-secured lacrimal caruncle implant can be used in implantations where the proximal end of the implant is exposed through the lacrimal caruncle or covered with tissue.

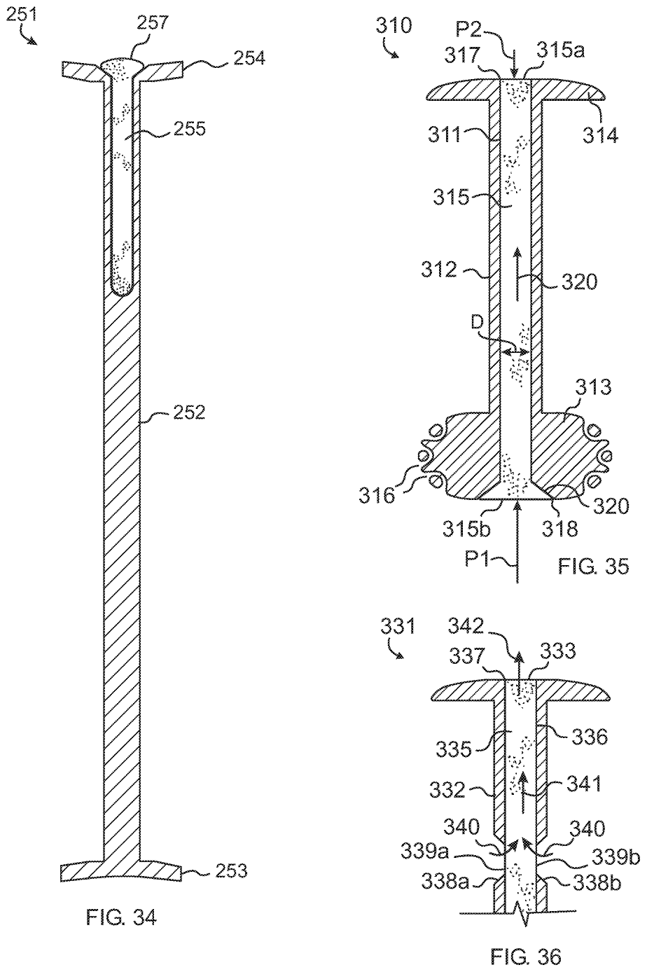

Referring now to FIG. 34 there is described an exemplary embodiment of a drug-releasing lacrimal caruncle implant 251 having an axially extended trunk portion 252 that allows a distal root 253 to bear against the lateral nasal wall to secure the implant from inadvertent extrusion. This embodiment is similar to the one shown in FIG. 28 but without the axially medial burl. Further, the proximal cap 254 can be adapted to provide the drug-carrying depot 255 with an expanded substantially conical proximal flare allowing the exposed proximal end 257 of the depot to have a larger exposed circular surface area to enhance the flow and reduce the risk of debris clogging the window.

Referring now to FIG. 35, there is shown an exemplary embodiment of a drug-releasing lacrimal caruncle implant 310 that can include a drug-releasing depot 315 carried within a cavity 311 extending axially within the trunk 312 of the implant to allow a flow-through condition to facilitate an increased and more predictably uniform flow of drug out of the implant. Similar to the embodiment of FIG. 25, the oblong trunk can terminate at a proximal cap 314 and at a distal root 313. The root can have a number of ingrowth channels 316 to help the implant avoid inadvertent extrusion, and thereby further secure the position of the implant when it extends inferomedially from the lacrimal caruncle.

This embodiment shows that the implant 310 can have an internal cavity 311 that has at least two windows 317,318 that expose separated surface portions 315a,315b of the depot 315 to the ambient environment outside the implant. Similar to the embodiment of FIG. 4, the first window 317 can be located at a proximal end of the implant to expose a proximal surface portion 315a of the depot. The second window 318 can form an opening through the body of the implant to expose a second surface portion 315b of the depot spaced an axial distance apart from the proximal surface portion. The presence of the windows can thus create a pathway for ambient body liquid to flow through the depot. The first, proximal window 317 can form an outlet while the second, distal window 318 can form an inlet spaced apart from the outlet. By locating the inlet more deeply within the tissues extending inferomedially from the lacrimal caruncle, a pressure differential can be created where the pressure P1 acting on the inlet is greater than the pressure P2 acting on the outlet. In this way the pressure differential between the two pressures P1 and P2 can drive a flow 320 of ambient body liquid into the inlet, through the depot, and out of the outlet, and on toward the eye. This embodiment shows that locating the windows at the opposite ends of the depot can tend to maximize the pressure differential.

The physical dimensioning of the depot can be selected to predictably adjust the flow rate according to known fluid dynamics principles. For example, the diameter D of a cylindrically shaped depot cavity can be selected to adjust flow rate according to the Hagen-Poiseuille equation:

$$F = \frac{π ( P 1 - P 2 ) {( D / 2 )}^{4}}{8 vL}$$

Where:
F is the volumetric flow rate;
D is the diameter of cavity;
v is the viscosity of the liquid; and,
L is the length of the cavity.

Thus embodiment also shows that the distal window 318 can have a substantially conical distal flare 320 while the proximal window 317 can be shaped in absence of such a flare thereby increasing the surface area of the inlet exposed to the greater pressure P1 and promoting an increase in the flow rate over an inlet lacking such a flare. The distal flare also provides the distal end with a larger exposed circular surface area to enhance the flow and reduce the risk of debris clogging the window. Alternately, the proximal window can be formed with such a flare for similar reasons.

Alternately, referring now to FIG. 36, there is shown an exemplary embodiment of a drug-releasing lacrimal caruncle implant 331 that can include a drug-releasing depot 335 carried within a cavity 336 extending axially within the trunk 332 of the implant to allow a flow-through condition to facilitate an increased and more predictably uniform flow of drug out of the implant. In this embodiment the first window 337 can be located at a proximal end of the implant to expose a proximal surface portion 333 of the depot and forming a liquid outlet. One or more additional windows 338a,338b can be provided in other parts of the implant body depending on the intended rate of flow of drug out of the depot.

In this embodiment the additional windows 338a,338b can be located to extend through the trunk 332 of the implant 331 to expose medial surface portions 339a,339b of the depot 335 that can be spaced an axial distance apart from the first, proximal window 337. In this way the additional windows through the trunk can form a liquid inlet to the depot allowing ambient body liquid to flow 340 through the additional windows, into the medial surface portions, to create a flow 341 through the depot, and a flow 342 out through the outlet and toward the eye.

In this way the above-described embodiments allow the entire ocular surface to be continuously coated with medication in a substantially uniform manner. The above-described embodiments can therefore allow maximum penetration of medication into the eye.

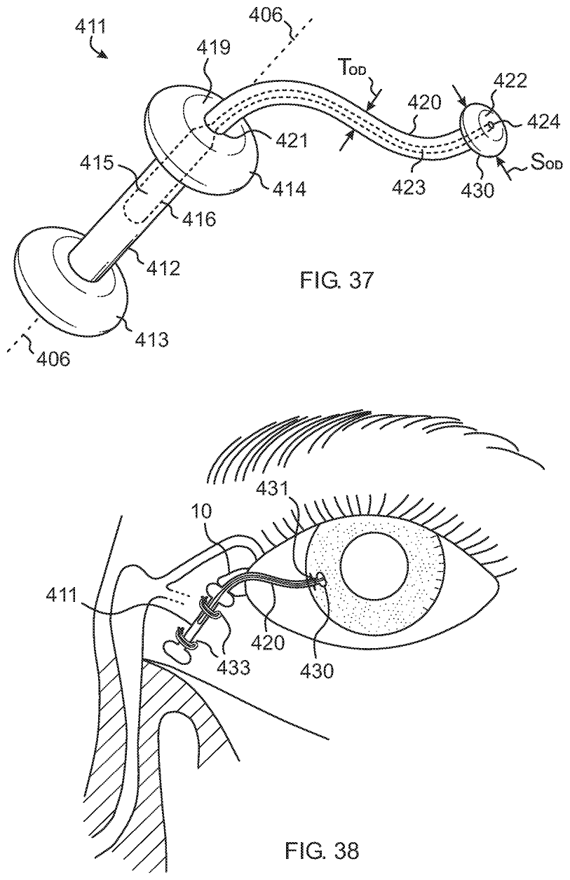

Referring now to FIGS. 37-38, there is shown an exemplary embodiment of a drug-releasing lacrimal caruncle implant 411 that can include a drug-releasing depot 415 carried within a cavity 416 extending axially within a trunk 412 elongated along an axis 406. Similarly to prior disclosed embodiments, the implant can include a root structure 413 connected to the distal end of the trunk, and a cap structure 414 connected to the proximal end of the trunk. The implant can also include a flexible discharge tube 420 extending from the proximal edge 419 of the cap to a securement structure 430 at its free end. The discharge tube can deliver a flow of drug from the depot through its central lumen 423 and out a proximal orifice 424 to a specific part of the eye anatomy to which the free end of the tube is secured.

For example, as shown in FIG. 38, the implant 411 can be emplaced through the lacrimal caruncle 10 in a manner similar to embodiments disclosed earlier. Sutures 433 can be used to ensure precise positioning of the body of the implant. The securement structure 430 at the free end of the flexible discharge tube 420 can be inserted through a small opening 431 in the cornea and into the anterior chamber of the eye. Medication can then flow from the depot 415 directly to the anterior chamber. In this way a predictable flow of medication can be established directly into specific locations of the eye that can benefit from direct exposure to medication without dilution.

The discharge tube 420 can be an oblong flexible hollow structure having a distal end 421 connected to the implant 411 at a proximal edge 419 of the proximal cap 414, and a free proximal end 422 terminating at the enlarged securement structure 430 which can help secure the free proximal end to the intended anatomical tissue by resisting unintentional extraction.

The discharge tube 420 can be made of a biocompatible flexible material such as silicone in order provide enough flexibility to conform to the eye globe and allow eye movement. The tube can be made large enough to carry an adequate flow of medication but small enough maintain comfort. Thus, the tube can have an outside diameter T_{OD} of between about 0.1 mm and 3.0 mm, and for most applications, more preferably between about 0.3 mm and 0.7 mm, and most preferably substantially 0.5 mm. The tube can have a length from distal end 421 to proximal end 422 of between about 2 mm and 20 mm, and for most applications, more preferably between about 4 mm and 8 mm, and most preferably substantially 6 mm. The diameter of the lumen 423 can be selected to adjust the flow rate and capillarity of the flow therethrough depending on the viscosity of liquid carrying the medication. Typically the diameter will be between ½ to ⅞ the outside diameter of the tube.

The securement structure 430 can have a radially enlarged ovoid shape having a widest outside diameter S_{OD}, taken substantially perpendicular to the elongation axis of the tube, of between about 2 mm and 20 mm for most applications, more preferably between about 2 mm and 10 mm, and most preferably between substantially 4 mm and 6 mm for most applications. Alternately, the securement structure can have various other geometrically bulbous shapes such as a sphere, ellipsoid, disk, or barb shaped.

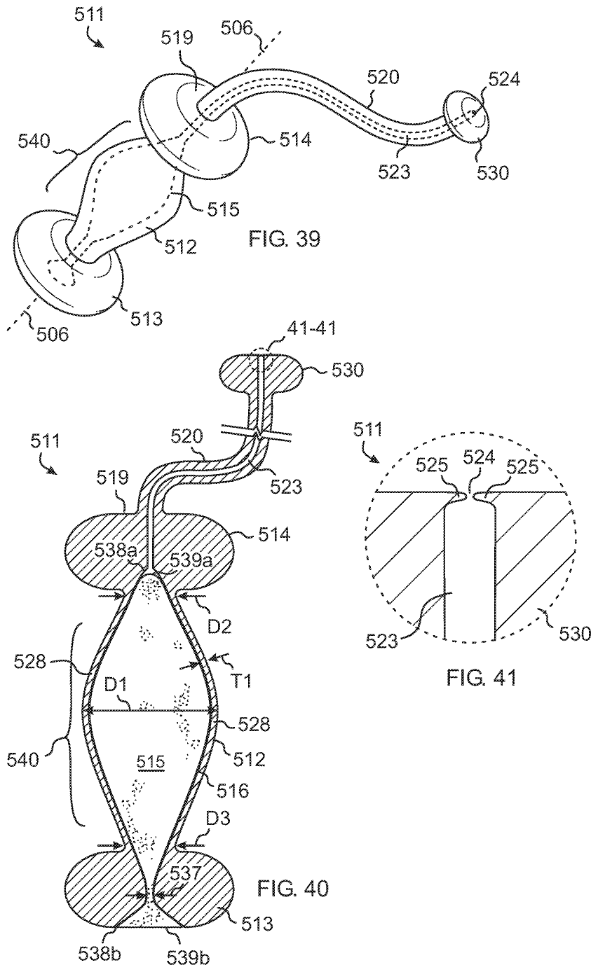

Referring now to FIGS. 39-45 there is shown an exemplary embodiment of a drug-releasing lacrimal caruncle implant 511 where the trunk 512 can be further adapted to form a manually collapsible bladder enclosing a drug-releasing depot 515. Once emplaced through the lacrimal caruncle 10 in a manner similar to earlier disclosed embodiments, collapsing the bladder and those a bulbous portion of the depot can cause a predictable and temporary flow of medication out of the depot. In this way the implant can provide a mechanism for pumping out a higher volume of liquid medication out of the depot in a shorter time.

As shown in FIGS. 39-40, the pumpable implant 511 can include the drug-releasing depot 515 carried within a cavity 516 extending axially within the trunk 512 which can be elongated along an axis 506. Similar to prior disclosed embodiments, the implant can include a root structure 513 connected to the distal end of the trunk, and a cap structure 514 connected to the proximal end of the trunk. The implant can also include a flexible discharge tube 520 extending from the proximal edge 519 of the cap to a securement structure 530 at its free end. The discharge tube can deliver a flow of drug from the depot through its central lumen 523 and out a proximal orifice 524 to a specific part of the eye anatomy to which the free end of the tube is secured. The root, trunk, cap, and discharge tube can be made from a unitary piece of resiliently flexible material such as silicone.

The trunk 512 of the pumpable implant can be shaped and dimensioned to have a radially bulbous region 540. For an implant having an axially symmetric trunk, the bulbous region can have a a substantially pointed-end ellipsoidal shape elongated along its major axis. In other words the bulbous region can have a cross-sectional diameter D1 that is largest at an axially medial location but which gradually tapers to smaller diameters D2,D3 at the proximal and distal ends of the trunk respectively. The cavity 513 inside the trunk can have a similar but dimensionally reduced shape so that the sidewall 528 formed between the cavity and the outer surface of the trunk have a substantially uniform thickness T1 which allows the resiliently flexible trunk to act as a bladder containing the drug-carrying depot 515. The thickest part of the bulbous region can be selected to be equidistant from the ends of the trunk. The use of both the radially widened cap and root structures straddling the bulbous region can help maintain the position of the implant during the rigors of repeated manipulation of the pumping action.

The cavity 513 can extend axially in a manner similar to the embodiment of FIG. 35 to have a first proximal window 538a exposing a proximal surface portion 539a of the depot 515 to the lumen 523 of the discharge tube 520, and a second distal window 538b exposing a distal surface portion 539b of the depot to the ambient environment outside the pumpable implant 511 at the distal end of the root 513. In this way the extended cavity provides the depot with a distal liquid inlet at the distal surface portion and a proximal liquid outlet at the proximal surface portion. The cavity can be further shaped to include an optional choke point 537 where the diameter of the cavity is reduced to a size less than or equal to the diameter of the discharge tube lumen. In this way flow of liquid into the depot from the distal window can be better regulated during the pumping and recovery cycle as described below.

In addition, as shown most clearly in FIG. 41, the pumpable implant 511 can also include one or more resiliently flexible flaps 525 extending radially inwardly from the periphery of the discharge tube orifice 524 at the proximal end of the securement structure 530. The action of the flaps can act similarly to a one-way cardiac valve where flow out of the orifice tends to bend the flaps outwardly encouraging flow whereas any flow inwardly is discouraged by the flaps blocking part or all of the orifice. In this way flow of liquid out of the lumen 523 can be better regulated during the pumping and recovery cycle as described below.

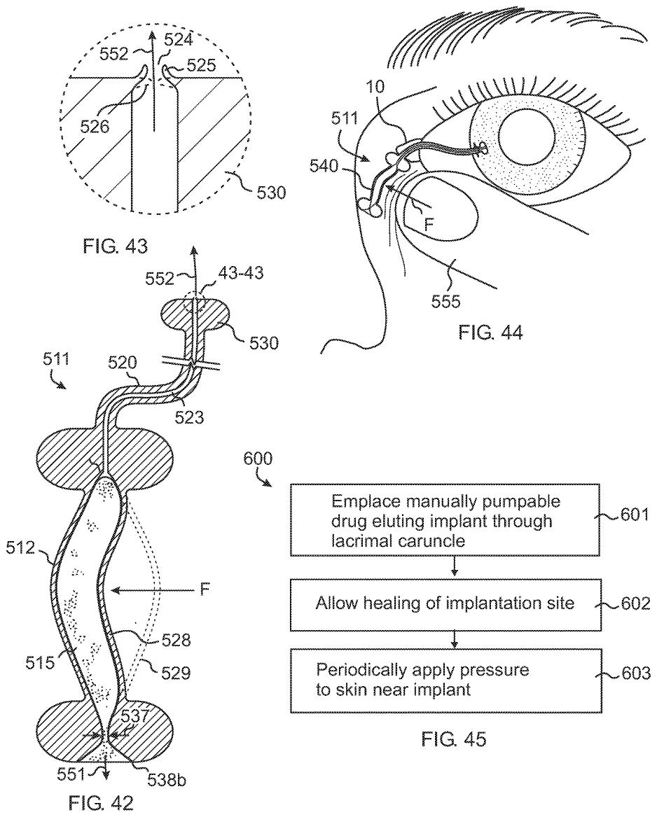

Referring now to FIGS. 42-44, the pumpable implant 511 is shown during a pumping process where a force F is applied radially to the bulbous region 540 or bladder of the trunk 512 thereby collapsing a portion of the resiliently flexible sidewall 528 from its non-collapsed state 529 to its collapsed state. This compresses the cavity 513 and the contained depot 515 to increase the pressure of the liquid residing in the depot. This causes a pair of simultaneous flows 551,552 of liquid out of the depot.

During compression of the bladder 540, the distal flow 551 out of the distal window 538b can have a very small flow rate due to the influence of the choke point 537. Simultaneously, the proximal flow 552 out of the proximal window 538a, through the lumen 523 of the discharge tube 520 and out of the proximal orifice 524 of the securement structure 530 can have a larger flow rate relative to that of the distal flow due to the influence of the wider diameter lumen and the opening of the flexible flaps 525 from their closed position 526.

After the bladder 540 is compressed the pumping action is complete. The force F on the bladder is discontinued and the recovery part of the cycle is allowed to commence. The resiliency of the sidewall 528 causes the bladder slowly return to its uncompressed state 529 as liquid is drawn into the depot 515. The flaps 525 on the orifice 524 resiliently return to their closed position 526 preventing little if any backflow through the orifice. On the other hand a small reverse flow through the distal window 538b and choke point 537 can slowly refill the depot over time.

As shown in FIG. 44, the force F which collapses the bladder 540 of the implant 511 during a pumping action can be applied by a patient's finger 555 tissues proximate to the lacrimal caruncle 10. In this way delivery of high volume liquid medication to the anterior chamber can occur outside of the physician's office.

Referring now to FIG. 45 there is shown a method 600 for emplacing and operating a manually pumpable drug-releasing implant into the tissues proximate to the lacrimal caruncle. The implant should be placed 601 so that the bulbous region rests transverse to the skin so that pressure applied to that region will cause the bulbous region to collapse. In other words the major axis of the implant can be oriented substantially parallel to the surface of the skin. In yet other words, the curve of the skin can be substantially tangent to the thickest part of the bulbous region. Further, the force can be applied in a direction substantially normal to the thickest part of the bulbous region.

After implantation 601 the site is allowed to heal 602. High liquid volume delivery of medication can then be accomplished by applying a force to the skin adjacent to the bulbous region and collapsing it. During recovery liquid in the depot can be given time to dissolve lyophized drug carried in the depot prior to the next periodic pumping action.

It will be readily appreciated by those skilled in the art that one or more features described in connection with one or more embodiments can be easily utilized by other embodiments.

While the exemplary embodiments of the invention have been described, modifications can be made and other embodiments may be devised without departing from the spirit of the invention and the scope of the appended claims.

What is claimed is:

1. A method for delivering a drug to orbital or periorbital tissues including the eye to treat a condition of a patient, said method comprises:
forming a tract through tissues proximate to the lacrimal caruncle of a patient;
placing an implant into said tract,
selecting said implant to have:
a body including a drug-carrying depot comprising:
a drug selected to treat said condition;
releasing said drug out of said implant and into said tissues;
wherein said placing comprises:
positioning said implant so that a proximal end of the implant is directly exposed to the lacrimal caruncle.

2. The method of claim 1, wherein said forming comprises:
further forming said tract through the lacrimal caruncle.

3. The method of claim 1, wherein said forming comprises:
further forming said tract through conjunctiva or other tissues surrounding the lacrimal caruncle, or the medial or inferomedial conjunctiva.

4. The method of claim 1, wherein said forming comprises:
extending said tract inferomedially from said lacrimal caruncle, surrounding conjunctiva, semilunar fold and adjacent tissues.

5. The method of claim 1, wherein said placing further comprises:
positioning said implant so that an exposed surface of the drug-carrying depot is directly exposed upon an outer surface of the lacrimal caruncle.

6. The method of claim 1, wherein said placing further comprises:
closing an opening in said lacrimal caruncle over a proximal end of said impact;
whereby said implant is fully surrounded by tissue.

7. The method of claim 1, wherein said forming further comprises:
excising the lacrimal caruncle to form a tract through said lacrimal caruncle; and,
dissecting said tract inferomedially from said lacrimal caruncle using a blunted instrument.

8. The method of claim 1 which further comprises:
allowing secretions emanating from said tissues to carry an amount of said drug out of said implant and onto the surface of the eye.

9. The method of claim 1, which further comprises:
said drug being a first drug;
assessing whether said first drug has been in any way effective; and,
recharging the implant with an amount of a second drug while said implant remains in vivo.

10. The method of claim 9, wherein said first drug is different from said second drug.

11. The method of claim 9, wherein said recharging comprises:
injecting a flow of said second drug from a syringe into said depot.

12. The method of claim 9, wherein said recharging comprises:
said depot being a first depot;
said first depot being removable from a remainder of said implant remaining in vivo;
replacing said first depot with a second depot containing said amount of said second drug.

13. The method of claim 1, which further comprises:
allowing said implant to fully biodegrade within said tract.

14. The method of claim 1, wherein said implant consists of one or more biodegradable and/or bioabsorbable materials.

15. The method of claim 1, wherein said implant comprises one or more biodegradable and/or bioabsorbable materials.

16. The method of claim 15, wherein said depot comprises one or more biodegradable and/or bioabsorbable materials, while a remainder of said implant comprises one or more non-biodegradable and/or non-bioabsorbable materials.

17. The method of claim 1, which further comprises:
retaining said implant in said tract during delivery of said drug onto said eye;
wherein said retaining occurs in absence of fixating said implant to the tissues surrounding the lacrimal caruncle with a clamp or other device apart from said implant.

18. The method of claim 1, which further comprises:
retaining said implant in said tract during delivery of said drug onto said eye;
wherein said retaining comprises providing said implant with a:
an oblong axial trunk;
a root located at a distal end of said trunk, said root having a radial diameter larger than an outside diameter of said trunk; and,
a cap located at a proximal end of said trunk.

19. The method of claim 18, wherein said placing comprises:
locating said root at least 7.0 mm from a surface of said lacrimal caruncle.

20. The method of claim 18, wherein said placing comprises:
locating said root between 0.5 to 30 mm from a surface of said lacrimal caruncle.

21. The method of claim 18, wherein said forming comprises:
extending said tract inferomedially from said lacrimal caruncle;
pushing a needle inferomedially through to the nasal cavity; and,
wherein said placing comprises:
locating said root adjacent to bone.

22. The method of claim 18, wherein said retaining further comprises providing said implant with a plurality of ridges formed onto at least one of said trunk and said root.

23. The method of claim 18, wherein said retaining further comprises providing said implant with at least one channel formed onto said root.

24. The method of claim 18, wherein said retaining further comprises providing said implant with said cap having a radially expanded flange, and a layer of adhesive formed onto a distal surface of said flange bearing against an outer surface of said lacrimal caruncle.

25. The method of claim 1, which further comprises:
providing said implant with a window thereby exposing a surface of said depot to fluids in liquid communication with the eye.

26. The method of claim 25, which further comprises:
providing said implant with a dimensionally expanded button extending outwardly beyond said window;
selecting a surface area for said button to adjust a flow rate of drug out of said depot.

27. The method of claim 25, which further comprises:
providing said implant with an opening in said body to a surface of said depot;
wherein said opening is located apart from said window.

28. The method of claim 1, which further comprises driving a flow of ambient body liquid through said depot between an inlet of said depot and an outlet of said depot, wherein said outlet is spaced apart from said inlet.

29. The method of claim 28, which further comprises adjusting a diameter of a cavity containing said depot to control a rate of flow through said depot.

30. The method of claim 28, which further comprises pumping a flow of said drug out of said implant.

31. The method of claim 30, wherein said pumping comprises:
providing said implant with a resiliently collapsible bladder containing a bulbous portion of said depot;
collapsing a portion of said bladder thereby temporarily increasing a pressure of liquid in said depot.

32. The method of claim 31, wherein said collapsing comprises:
pushing a finger against the skin proximal to said bladder.

33. An implant for delivering a drug to orbital tissues including the eye to treat a condition of a patient, said implant comprises:
a body;
a drug-carrying depot comprising a drug;
said implant being shaped and dimensioned to be inserted inferomedially through a tract in the lacrimal caruncle of the patient;
whereby while the implant is located in the lacrimal caruncle of the patient, a surface of said drug-carrying depot is directly exposed to fluids in liquid communication with the eye.

34. The implant of claim 33, wherein said body further comprises:
an oblong axial trunk;
a root located at a distal end of said trunk, said root having a radial diameter larger than an outside diameter of said trunk; and,
a cap located at a proximal end of said trunk.

35. The implant of claim 34, which further comprises:
a window through said cap exposing a proximal end of said depot.

36. The implant of claim 35, wherein said window is oversized creating a peripheral gap between said window and said proximal end.

37. The implant of claim 35, wherein said proximal end of said depot comprises:
a dimensionally expanded button extending out beyond said window;
wherein a surface area of said button is selected to adjust a flow rate of drug out of said depot.

38. The implant of claim 34, which further comprises:
a cavity located within said body, wherein said cavity is shaped and dimensioned to secure said depot therein.

39. The implant of claim 34, which further comprises:
said oblong axial trunk having an axial length of between 0.5 and 30 mm;
said outside diameter of said trunk being between 0.01 and 7 mm; and,
said radial diameter being between 0.1 and 12 mm.

40. The implant of claim 33, which further comprises at least one prominence extending laterally from one of said trunk and root.

41. The implant of claim 40, wherein said prominence has a first axially perpendicular cross-sectional dimension greater than a second axially perpendicular cross-sectional dimension of said trunk.

42. The implant of claim 33, which further comprises a plurality of axially spaced apart prominences extending radially from said body.

43. The implant of claim 33, which further comprises a flange extending laterally at a proximal end of said body.

44. The implant of claim 43, wherein said flange comprises a distal flange surface oriented to rest against at least part of the tissue surrounding an opening of said tract when said implant is properly emplaced through said lacrimal caruncle.

45. The implant of claim 44, which further comprises a layer of biocompatible adhesive located on said distal flange surface.

46. The implant of claim 34, which further comprises a burl extending radially from a medial portion of said trunk.

47. The implant of claim 33, which further comprises:
a plurality of spaced apart windows through outer surfaces of said implant exposing surfaces of said depot.

48. The implant of claim 33, wherein said trunk comprises:
a resiliently collapsible bladder containing a bulbous portion of said depot;
whereby collapsing a portion of said bladder pumps a flow of drug out of said implant.

49. The implant of claim 33, wherein said body comprises a first non-biodegradable and/or non-bioabsorbable material and said depot comprises a first biodegradable and/or bioabsorbable material.

50. The implant of claim 33, wherein said body comprises a first biodegradable and/or bioabsorbable material and wherein said depot comprises a second biodegradable and/or bioabsorbable material.

51. The implant of claim 50, wherein said first biodegradable and/or bioabsorbable material is selected from the group consisting of:
poly (lactic co glycolic acid) (PLGA);
hydroxymethylcellulose;
collagen;
polydioxanone;
E-Caprolactone-L-Lactide-copolymer;
polycaprolactone (PCL)/PLGA; and,
polyethylene glycol (PEG).

52. The implant of claim 33, wherein said body further comprises biodegradeable particles selected from the group consisting of:
colloidal particles, liposomes, microparticles, nanoparticles and nanospheres

53. The implant of claim 33, wherein said drug is dispersed in biodegradable polymeric matrix or membrane.

54. The implant of claim 33, wherein said drug comprises a therapeutic agent selected from the group consisting of:
bimatoprost, latanoprost, latanaprostene bunod, netarsudil, tafluprost, travoprost, brinzolamide, betaxolol, carteolol, levobunolol, timolol, apraclonidine, brimonidine, ganciclovir, acyclovir, famcyclovir, gentamicin, tobramycin, moxifloxacin, levofloxacin, ocufloxacin, ciprofloxacin, sulfacetamide products, polymyxin, neomycin, penicillin, cephalosporins, doxycycline, tetracycline, minocycline, erythromycin, biaxin, trifluridine, dexamethasone, triamcinolone, fluocinolone, and cyclosporine.

55. The implant of claim 33, wherein said drug comprises a therapeutic agent selected to treat dry eye syndrome.

56. The implant of claim 33, wherein said body is coated with a biodegradeable material.

57. The implant of claim 33, wherein said drug is conjugated to a biodegradeable material.

58. The implant of claim 33, wherein said implant establishes a flow of drug-containing fluid of at least 0.000001 milliliter per minute to said eye.
